(19) United States
(12) Patent Application Publication
(10) Pub. No.: US 2022/0319689 A1
(43) Pub. Date: Oct. 6, 2022

(54) SYSTEMS AND METHODS FOR IMPROVED INTERFACE NOISE TOLERANCE OF MYOELECTRIC PATTERN RECOGNITION CONTROLLERS USING DEEP LEARNING AND DATA AUGMENTATION

(71) Applicant: Rehabilitation Institute of Chicago d/b/a Shirley Ryan Abilitylab, Chicago, IL (US)

(72) Inventors: Yuni Teh, Chicago, IL (US); Levi Hargrove, Chicago, IL (US)

(21) Appl. No.: 17/709,133

(22) Filed: Mar. 30, 2022

Related U.S. Application Data
(60) Provisional application No. 63/168,076, filed on Mar. 30, 2021.

(51) Int. Cl. G16H 40/63; G06N 3/08; G16H 50/70

(52) U.S. Cl. CPC G16H 40/63; G06N 3/08; G16H 50/70

(57) ABSTRACT

A system includes an input device in operable communication with a processor. The input device generates a plurality of signals representing an intended control of a prosthesis. The processor executes a predetermined pattern recognition (PR) control method that applies the plurality of input signals to at least one machine learning model trained using a training data set augmented with synthetic noise. The at least one machine learning model is configured to align the plurality of input signals to a low-dimensional manifold defining features and classify the features to identify a command for moving the prosthesis. The predetermined PR control method leverages the deep learning of the at least one machine learning model and the augmented training data to improve noise tolerance.

### CROSS REFERENCE TO RELATED APPLICATIONS

The present application claims the benefit of U.S. Provisional Patent Application No. 63/168,076 filed on Mar. 30, 2021 and entitled “LATENT SPACE METHODS AND SYSTEMS FOR MYOELECTRIC CONTROL,” which is hereby incorporated by reference in its entirety.

### FEDERALLY SPONSORED RESEARCH OR DEVELOPMENT

The present invention was made with government support under National Institutes of Health (NIH) award no. R01HD094861. The government has certain rights in the invention.

### FIELD

The present disclosure generally relates to prosthetic and assistive devices; and in particular, to improvements in latent space methods for myoelectric control.

### BACKGROUND

Clinically available myoelectric pattern recognition systems for upper limb prostheses use surface electrodes to measure electromyographic (EMG) signals. The accuracy of these controllers relies on repeatable and separable muscle activation patterns. Unfortunately, EMG signals measured from surface electrodes can be unstable under various real-world conditions.

For instance, surface electrodes are prone to signal noise caused by electrode lift-off and wire breakage. Electrode lift-off commonly occurs due to factors such as residual limb volume changes and ill-fitting sockets. It corrupts the measured muscle patterns and consequently degrades control performance. Additionally, regular prosthesis usage can cause wire breakage. Based on data from a take home study, broken wires were a common reason which impaired control performance, emphasizing the clinical significance of this problem.

Researchers have sought to resolve the effects of electrode noise through signal processing, spatial filtering, data fusion, and recalibration techniques. Of these methods, the fault detection and fast recalibration approach offered the most complete implementation. However, since recalibration only occurred when a disturbance was detected, this method was highly dependent on the fault detector, which missed approximately 30% of the disturbances. Thus, there is still a need for an effective and clinically feasible noise-resistant control system.

It is with these observations in mind, among others, that various aspects of the present disclosure were conceived and developed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is an illustration of an experimental device and setup associated with an exemplary first system for myoelectric control of an intact limb that leverages the inventive concept of applying data augmentation and deep learning techniques to improve interface noise tolerance.

FIG. 1B is an illustration of an experimental device and setup associated with the first system for myoelectric control for an amputated limb.

FIG. 2A is an illustration showing four limb positions that subjects were instructed to cycle through for data collection associated with the first system as described herein.

FIG. 2B is a graphical representation showing examples of synthetically corrupted data generated by flatlining each EMG channel for data collection used by the first system.

FIG. 3 is a diagram illustrating data flow for learning a mapping between surface EMG signals and a low-dimensional subspace to classify myoelectric signals for the first system.

FIGS. 4A-4B are respective diagrams showing an architecture for a supervised denoising variational autoencoder of FIG. 3 according to the first system.

FIGS. 5A-5B are graphical representation respectively showing classification accuracies for intact-limb subjects and amputee subjects according to the first system.

FIG. 6 is a simplified block diagram of an exemplary second system for myoelectric control of intact limb participants that leverages the inventive concept of applying data augmentation and deep learning techniques to improve interface noise tolerance.

FIG. 7A is an illustration of an experimental device and setup associated with the second system for myoelectric control of intact limb participants.

FIG. 7B is an illustration of an experimental device and setup associated with the second system for amputee participants.

FIG. 7C is a graphical illustration of a virtual reality environment used for data collection associated with the second system.

FIG. 8A is an illustration of completed wrist and hand gestures in four arm positions associated with the second system.

FIG. 8B is an illustration of a network architecture of a multilayer perceptron (MLP) associated with examples of the second system.

FIG. 8C is an illustration of a network architecture for a convolutional neural network (CNN) associated with examples of the second system.

FIG. 8D is an illustration of a network architecture for an MLP-linear discriminant analysis (LDA) and CNN-LDA control strategy that uses one or more neural networks to compute EMG latent features that were classified by LDA classifiers as described herein.

FIG. 9 is an illustration of examples from a real noise database associated with the second system as described herein fused with a clean EMG signal

FIG. 10A-10B are graphs illustrating average classification accuracies and differences from baseline LDA accuracies for intact limb participants.

FIGS. 11A-11B are graphs illustrating average classification accuracies and differences from baseline LDA accuracies for amputee participants.

FIG. 12 is an illustration of three-dimensional latent representations of AMP5's training and test data sets using LDA components related to the second system as described herein.

FIG. 13 is a simplified block diagram of an exemplary method for data augmentation and deep learning techniques to improve interface noise tolerance of myoelectric pattern recognition controllers.

FIG. 14 is a simplified illustration of an exemplary computing device which may be implemented to execute functionality described herein.

Corresponding reference characters indicate corresponding elements among the view of the drawings. The headings used in the figures do not limit the scope of the claims.

### DETAILED DESCRIPTION

Aspects of the present disclosure relate to various examples of a system and associated methods for implementing data augmentation and deep learning techniques to improve interface noise tolerance of myoelectric pattern recognition (PR) controllers which may be used for myoelectric control of a device by decoding user intent from muscle signals.

In one embodiment, an array of surface electromyography (EMG) electrodes measures diffuse muscle signals that include redundant neural information. The system learns a mapping between surface EMG signals and a low-dimensional subspace to capture only the features that are most salient for signal reconstruction and intent recognition. This in effect denoises signals and reduces the impact of signal non-stationarities, thereby improving controller robustness. In some embodiments, the subject approach leverages the redundancies in decoded neural information across multi-channel EMG signals to build robust classifiers. This is based on the idea that an array of surface EMG signals measures diffuse muscle signals that contain overlapping neural information and, as such, can be compressed into a low-dimensional manifold without losing discriminative information.

In some embodiments, the system uses a supervised denoising variational autoencoder to process the EMG signals obtained from the EMG array. A variety of signals are measured by the EMG array across different conditions, which can be synthetically corrupted. The denoising variational autoencoder is then trained to 1) reconstruct original clean signals from their corrupted counterparts and 2) classify the reconstructed signals into their labeled classes. After the network is trained, an encoder module can be separated and used to map new signals to the learned latent manifold.

The present inventive concept is believed to be the first to use a signal corruptor with a denoising network on EMG signals, which in tandem give rise to a more robust latent representation. The system was envisioned to mitigate signal noise for upper limb prosthesis control; however, the system can be used for any EMG controlled device or application (e.g., lower-limb prostheses, exoskeletons, powered orthotics, robotic manipulators, video/computer games). In addition to mitigating noise, latent space methods can also be used to achieve simultaneous control and across-subject generalizability.

### First System Example (100)

### I. Introduction

In a first example of a system for improving interface noise tolerance of myoelectric pattern recognition controllers, designated system 100, the system 100, which includes at least one processor or processing element, is configured to learn latent representations of surface EMG signals that are robust to single channel signal corruption and to use this latent space to classify wrist and hand movements from clean and corrupted inputs. To that end, a deep learning model was implemented based on denoising variational autoencoders, which can extract latent distributions that are robust to noise by learning to reconstruct clean inputs from partially corrupted inputs. In developing the system 100, it was hypothesized that the latent space classifiers would yield higher accuracies than conventional LDA classifiers.

### II. Methods

A first study associated with the system 100 was approved by the Northwestern University Institutional Review Board. For the first study, fourteen subjects with intact limbs (ITL) (ages 20-29, eight male, six female) and six subjects with below-elbow amputations (AMP) (five male, one female) participated after giving written informed consent.

### A. Subject Setup

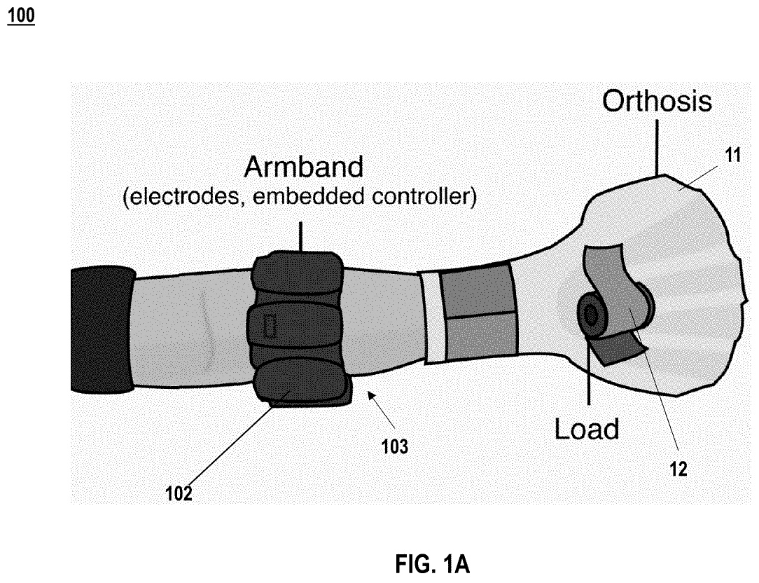

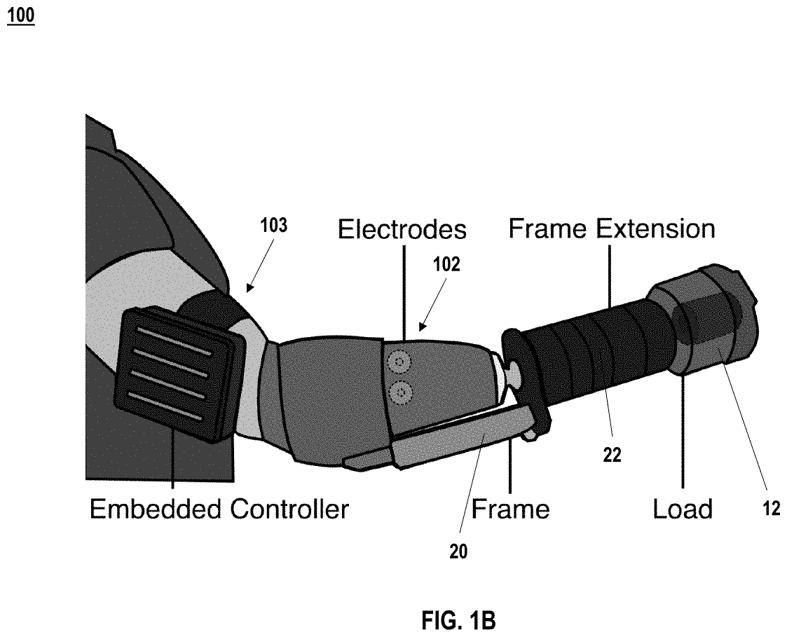

Intact Limb Subjects: Intact limb (ITL) subjects wore an armband around their right forearm that housed six pairs of stainless steel electrodes (Motion Control Inc.) that collectively form an EMG array 102 of the system 100. A forearm orthosis 11 was used to encourage isometric contractions. A 400 g weight load 12 was also attached to the orthosis to simulate the weight of a prosthesis (FIG. 1A).

Amputee Subjects: Six pairs of wet Ag/AgCl electrodes (Bio-Medical Instruments) were arranged to collectively form EMG array 102 circumferentially around each amputee (AMP) subject's residual limb and secured under a silicone liner. A frame 20 was strapped around the residual limb and attached to the liner with a magnet. A box that included a 400 g weight load 12 was affixed to the frame via extensions 22 that placed the load at the position of a terminal device (FIG. 1B).

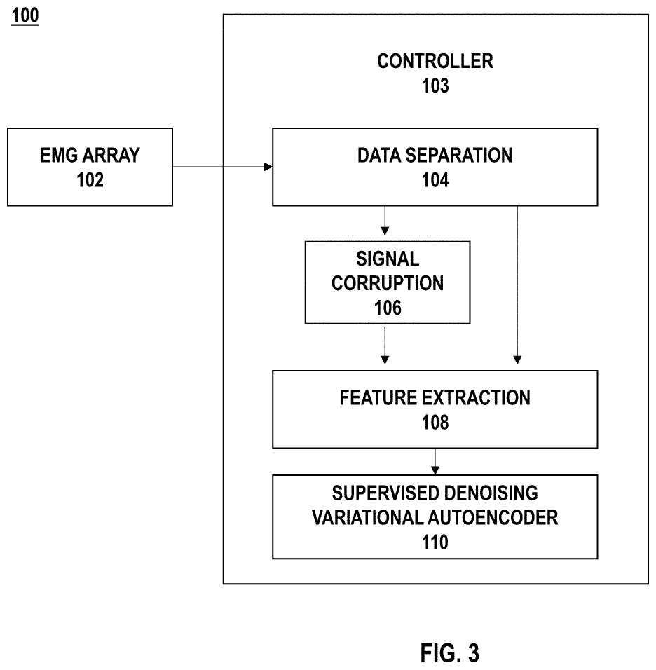

In some embodiments, the EMG array 102 communicates or is in operable communication with an embedded controller 103 that collects EMG data from the EMG array 102. As shown in FIG. 3, controller 103 facilitates data processing by the system 100 to decode user intent from the EMG data. In another embodiment, EMG data from EMG array 102 is collected and communicated by the controller 103 to an external device or cloud-based computing server for data and signal processing efforts or to perform several processes as will be discussed in further detail.

### B. Data Collection

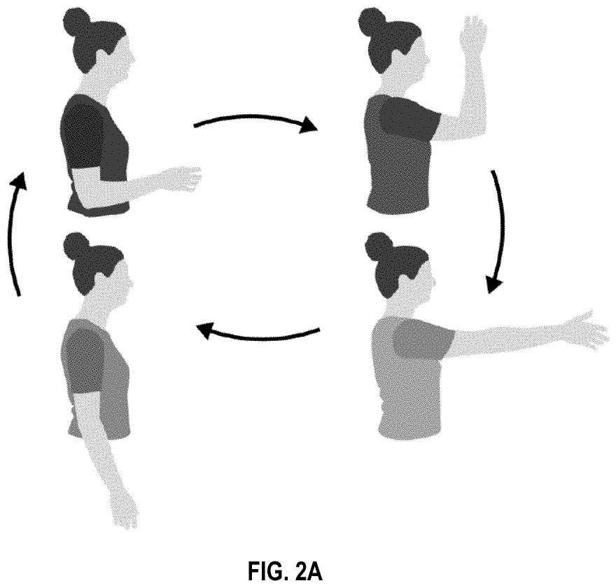

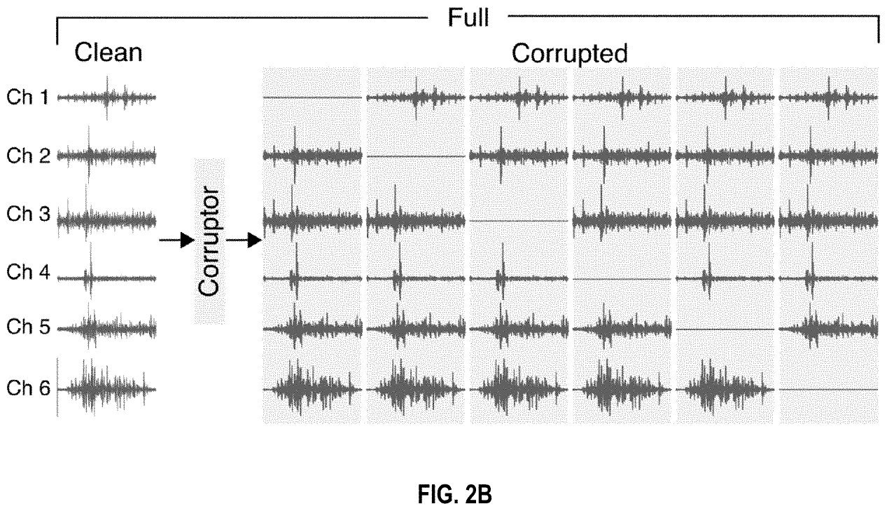

To simulate clinical training methods, subjects wearing the EMG array 102 moved their arms around in the workspace while performing wrist and hand gestures shown in FIG. 2A. All ITL subjects and two AMP subjects completed seven gestures (no motion, hand open/close, wrist pronation/supination, and wrist flexion/extension). The remaining five AMP subjects were unable to reliably perform wrist flexion/extension; hence, these subjects only used five gestures (no motion, hand open/close, and wrist pronation/supination). Each gesture was held for 2.5 s and repeated five times, resulting in 10s of data.

### C. Data Processing

In one embodiment, all signals obtained from EMG array 102 are sampled at 1 kHz, amplified with a hardware gain of 2, and band-pass filtered between 70-300 Hz. Signals are then collected in 200 ms windows with 25 ms increments.

Referring to FIGS. 2B-4B, the raw data from EMG array 102 is divided into training and testing subsets (70/30) using stratified sampling by a data separation module 104 executed or otherwise associated with controller 103. To simulate interface noise in both subsets, a signal corruptor module 106 flatlines each EMG channel one at a time, as shown in FIG. 2B. The data is separated into a ‘clean’ set (original signals), a ‘corrupted’ set (corrupted signals), and a ‘full’ set included both original and corrupted signals. Finally, four time-domain features are extracted by feature extraction module 108 to obtain mean absolute value, waveform length, zero crossings, and slope sign changes from each channel of the EMG array 102 to obtain a plurality of feature windows (6×4). The 6×4 2-dimensional feature windows are then used as input to a supervised denoising variational autoencoder 110.

### D. Supervised Denoising Variational Autoencoder

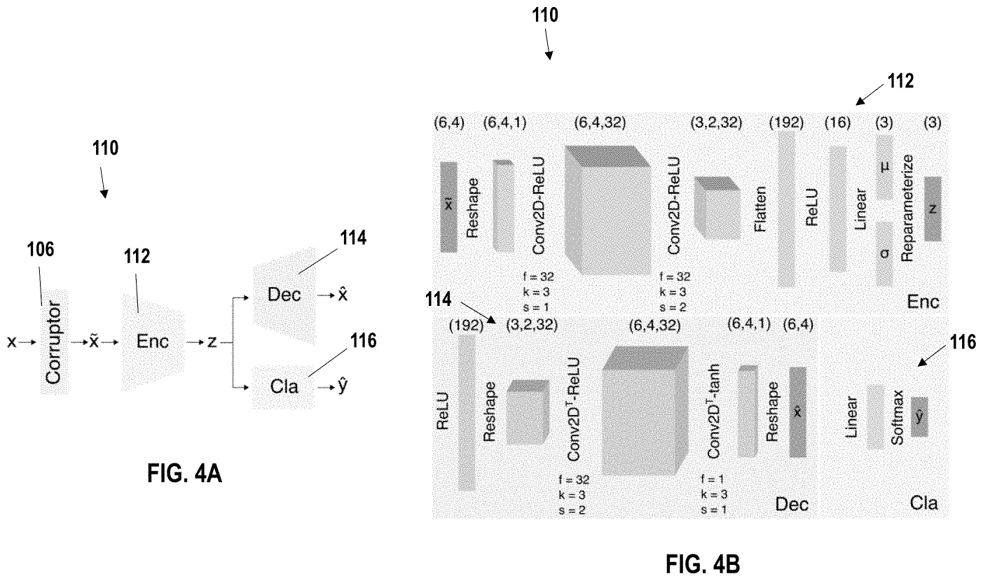

Using the full training dataset, the supervised denoising variational autoencoder (VAE) 110 is trained to reconstruct and classify the EMG feature windows extracted by the feature extraction module 108. Augmenting the training dataset with the corrupted subset by signal corruption module 106 enables the VAE 110 to learn latent representations that were robust to these signal disturbances. The VAE 110, illustrated in FIGS. 4A and 4B, includes three modules:

Encoder. An encoder 112 learns to project the input to a latent distribution parameterized by μ and σ, which is then used to sample a latent variable z. A latent dimensionality of 3 was selected for one embodiment, predicated on previous work that uncovered a 3-dimensional latent space that could explain approximately 97% of EMG variance. Convolutional layers are included to preserve the spatial relationships between channels.

Decoder. A decoder 114 learns to reconstruct the clean input x from the latent variable z. Decoder 114 includes transposed convolutional layers to expand the latent variables into the original features.

Classifier: A classifier module 116 is trained to predict movement classes ŷ from the latent variable z. This supervised classifier module 116 is included so that the VAE 110 would learn latent representations that are both reconstructable and linearly separable.

The VAE 110 is trained to minimize three losses: a mean squared error between the original, uncorrupted input x and a reconstructed input \hat{x}, the Kullback-Leibler divergence between the latent distribution N(μ, σ²) and the standard normal distribution N(0, 1); and the cross-entropy loss between the ground truth class labels y and the predicted class labels ŷ. All inputs are normalized to a range of −1 to 1 using MinMax scaling. In one implementation, an Adam optimization algorithm with a mini-batch size of 128 and 30 epochs is used.

It would be appreciated by one of ordinary skill in the art that features of the controller 103, and/or other aspects of the system 100 may be implemented as code and/or machine-executable instructions executable by a processor of a computing device that may represent one or more of a procedure, a function, a subprogram, a program, a routine, a subroutine, a module, an object, a software package, a class, or any combination of instructions, data structures, or program statements, and the like. In other words, one or more of the features of the controller 103 described herein may be implemented by hardware, software, firmware, middleware, microcode, hardware description languages, or any combination thereof. When implemented in software, firmware, middleware or microcode, the program code or code segments to perform the necessary tasks (e.g., a computer-program product) may be stored in a computer-readable or machine-readable medium (e.g., the memory of the computing device), and the computing device performs the tasks defined by the code.

### E. Classifier Evaluation

Four classification methods were evaluated by calculating their offline accuracies on the full, clean, and corrupted testing data sets. The Clean Linear Discriminant Analysis (LDA) and Full LDA methods were formulated on clinically available pattern recognition systems while the Aligned LDA and NN methods leveraged the latent space learned by the proposed VAE.

Clean LDA: The Clean LDA method used an LDA classifier that was trained on the clean training data set only. This strategy closely resembled the gold standard of clinically available pattern recognition controllers and thus acted as a baseline.

Full LDA: The Full LDA method used an LDA classifier that was trained on the full training data set, including clean and corrupted signals.

Aligned LDA: The Aligned LDA method used an LDA classifier that was trained on the full training data set aligned to the latent space with the trained encoder. The testing data set was also aligned prior to evaluation. In other words, this method classified the latent space representations using an LDA classifier.

Neural Network (NN): The NN method used the encoder and classifier modules from the VAE that was trained on the full training data set.

### F. Statistical Analyses

ITL and AMP data were analyzed separately. Accuracies between each classifier were compared using paired t-tests with Bonferroni corrections (α=0.05).

### III. Results

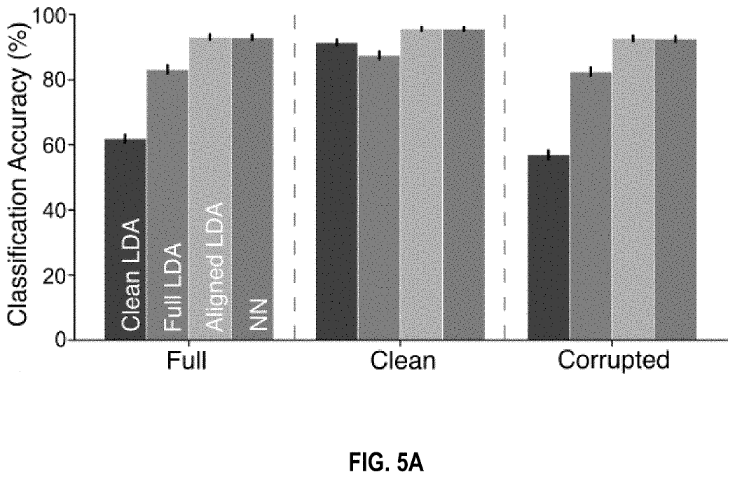

ITL and AMP subject results, displayed in FIGS. 5A and 5B respectively, generally followed the same trends.

When evaluated on the full testing set, the Clean LDA classifier performed significantly worse compared to all other methods (p<0.001). This was driven by its inability to accurately label corrupted data (ITL: 56.98±1.37%, AMP: 55.62±1.86%). It was considerably more accurate when classifying clean data (ITL: 91.44±0.93%, AMP: 86.51±3.25%), even yielding a significantly higher accuracy than the Full LDA classifier for ITL subjects (p<0.001).

Although the Full LDA classifier outperformed the Clean LDA classifier on the full and corrupted data sets, its accuracy was significantly lower than that of the latent space methods for all testing sets (p<0.001).

Across both subject populations and the three testing sets, the Aligned LDA and VAE classifiers outperformed the Clean LDA and Full LDA classifier (p<0.001), yielding similarly high accuracies that were not significantly different from each other (p=1). Notably, both latent space classifiers obtained higher accuracy rates on corrupted data than the Clean LDA classifier obtained on clean data (ITL: p<0.02, AMP: p<0.001).

### IV. Discussion

The system 100 and its description and testing herein shows that a denoising variational autoencoder can learn latent representations that are robust to single channel corruption. The results from the Clean LDA classifier showed that signal corruption in just a single channel can substantially diminish the accuracy of current gold standard control systems. Although the relationship between offline accuracy and real-time performance is not yet clearly understood, previous work suggests that an error rate discrepancy of this magnitude could cause controllability to drop from high to low.

Unsurprisingly, training an LDA classifier with both clean and corrupted signals improved its robustness to interface noise. However, with the average error rate of the Full LDA classifier hovering around 20%, it is unclear whether this training data augmentation method is sufficient for reliable real-time control. The efficacy of this strategy is contingent on the movement classes remaining linearly separable in the presence of all possible signal disturbances that occur throughout regular prosthesis usage.

Ultimately, the latent space methods were superior to conventional methods. The nonlinear activation functions and convolutional layers of the VAE facilitate the learning of more complex structures compared to the LDA-based techniques. Additionally, the modular nature of deep learning models allows them to minimize multiple loss functions concurrently. In this case, while LDA- and VAE-based methods both optimized separability, the VAE did so while also learning to denoise corrupted signals. As such, the VAE learned a low-dimensional representation that was both separable and robust to noise.

### Summary of System 100:

As described, a supervised denoising variational autoencoder learns low-dimensional representations of wrist and hand movements that are continuous, linearly separable, and robust to single channel noise. It was shown that this latent space can be used to build noise-resistant classifiers that are significantly more accurate than current state-of-the-art classifiers. These results are promising and motivate further development of this technique, which could reduce the frequency of controller recalibrations and improve prosthesis controller stability across practical situations.

### Second System Example (1000)

A second example of a system for improving interface noise tolerance of myoelectric pattern recognition controllers, designated system 1000, shall now be described including testing and discussion thereof.

### I. Background

As previously discussed, PR control methods measure EMG signals from an array of electrodes and learn the patterns of muscle activity that correspond to intended movements. Typically, PR controllers use a classifier to classify descriptive features extracted from windowed EMG signals. Use of linear discriminant analysis (LDA) classifiers is one example as they are computationally simple to train and implement. PR control has been shown to improve functional outcomes for prosthesis users, provided the EMG interface is stable and the signals are repeatable.

However, regular usage of myoelectric prosthesis gives rise to various sources of signal disturbances that degrade classification accuracy. Changes in residual limb volume, limb position, and socket loading can cause electrodes to intermittently lose contact with the skin. Signal abnormalities stemming from electrode or wire failures also occur with prolonged prosthesis use. Interface noise in just one channel is often detrimental to the accuracy of PR control strategies, rendering the device unusable until it can be recalibrated.

Previous studies have proposed several approaches to resolve the effects of interface noise. For example, signal processing algorithms can be used to denoise affected channels before classification. The generalizability of these methods is limited, however, as most focus on filtering out periodic noise (eg. electrical noise) and do not address the effects of intermittent noise signals. Another approach uses control strategies that adjust their classifier parameters to adapt to changes in EMG signals. Of note is a fast-retraining LDA method that detects and removes noisy channels before recalibrating its weights. Though they can increase classification accuracy, these adaptive control methods require additional processing steps during classification.

One promising solution (aspects of which are contemplated by the present inventive concept) exploits the signal redundancies across EMG channels to build a classifier that is inherently robust to interface noise. Since surface electrodes measure diffuse muscle activity, an array of surface EMG signals contains overlapping neural information. These redundant signals can therefore be compressed into a low-dimensional manifold that retains discriminative features and is less sensitive to input disturbances. Using this concept, spatial filtering, linear factorization, and data fusion can be employed to build robust classifiers. However, these examples used setups that are not yet clinically practical (eg. high-definition EMG arrays).

The features of the system 1000 described herein were developed upon the notion that to train an accurate PR controller, it is essential the training data typifies signals in real scenarios. For example, performing dynamic arm movements during training data collection instead of maintaining a static position significantly improves LDA classification performance across different limb positions. By increasing training data variability, classifiers are encouraged learn discriminative features that are consistent across sources of variance, thus preventing overfitting. However, it is a challenge to physically collect enough data to sufficiently represent realistic scenarios. To alleviate the burden of extensive training data collection, data augmentation can be used to artificially introduce variability in a systematic manner, as described herein according to aspects of the system 1000.

As the variance of the training data increases and the data become more complex, simple linear classifiers may not be equipped to adequately model hidden structures. Deep learning models are known for their ability to learn complex nonlinear relationships within large datasets. Specifically, deep encoders compress high-dimensional inputs into low-dimensional latent subspaces. A desirable characteristic of these manifolds is that large disturbances in the input have minimal effects on their latent representations. Depending on the model's loss function, the latent space can be optimized for a specific objective, such as reconstructing or classifying the input.

Convolutional neural networks (CNNs) are another useful deep learning tool and may be implemented by the system 1000 as described herein. CNNs are commonly used for image processing applications where the relative locations of pixels are crucial to the underlying structure. While sequential neural networks use flattened vectors as inputs, CNNs preserve spatial relationships by allowing multidimensional input matrices. Thus, CNNs are well-equipped to disentangle EMG signal redundancies, which are dependent on electrode locations.

One possible application of the system 1000 described herein is to provide a clinically feasible, noise-tolerant myoelectric PR controller that classifies hand and wrist movements. To that end, aspects of the system 1000 incorporate the use of data augmentation and deep learning techniques to uncover a latent subspace in which movement classes are separable and robust to interface noise. In a study of the system 1000 described herein, five control strategies were trained and their performances were evaluated on normal EMG data and EMG data that contained up to four channels of interface noise. These strategies included a conventional LDA algorithm, an adaptive LDA algorithm, an LDA algorithm trained with an augmented data set, an LDA algorithm that classifies latent EMG variables computed by a multilayer perceptron, and an LDA algorithm that classifies latent EMG variables computed by a CNN. It was hypothesized that the CNN control strategy would be the most accurate non-adaptive classifier across all noise conditions because it preserves spatial dependencies and contains nonlinearities.

### II(A). General Non-Limiting Features of the System (1000)

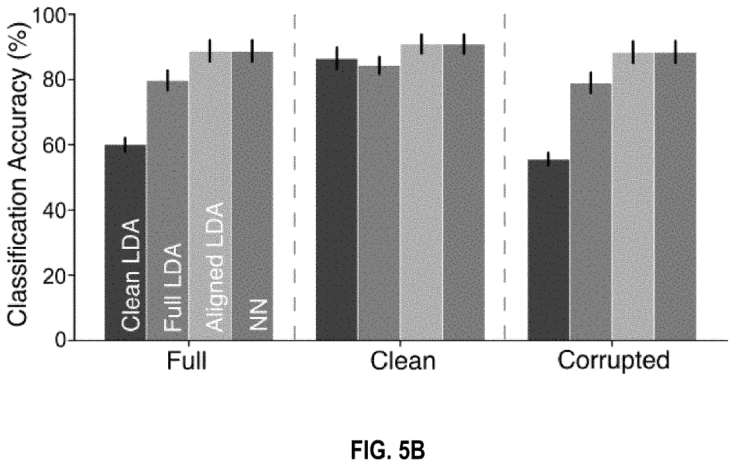

Referring to FIG. 6, the system 1000 generally includes at least one processor or processing element 1002 in operable communication with an input device 1004. The input device 1004 includes any device that generates and/or collects data associated with an intended movement by a patient. For example, the input device 1004 can provide a plurality of signals 1006 representing an intended or desired control 1008 of a prosthesis by the patient. In general, the processor 1002 applies the plurality of signals 1006 to a predetermined pattern recognition control protocol 1010 (or simply “controller” or “control strategy”) stored in memory (1003) to identify a command 1012 (via a classification 1013 or otherwise) for moving the prosthesis (or for some actuation of an assistive, rehabilitative, or virtual device). In one non-limiting example, the plurality of signals may be or include EMG data, and the input device 1004 can include an EMG array, however the system 1000 is not limited in this regard.

The predetermined PR control protocol 1010 generally defines at least one machine learning (ML) model, such as neural network, represented as ML model 1014A and ML model 1014B and defined collectively at least one ML model 1014, trained using a training data set 1016 augmented with synthetic noise 1018 in the various manners described herein. Executing the predetermined PR control protocol 1010, the processor 1002 is operable to align the plurality of input signals 1006 to a low-dimensional manifold defining features, and then to classify the features to identify a command 1020 (for moving the prosthesis or otherwise). In one example, the at least one ML model 1014 includes an LDA classifier that is trained with latent features of the training data set 1016, the training data 1016 set constructed by systemic corruption of a predetermined number of a plurality of channels of raw training signals from the input device 1004. The plurality of signals 1006 may define electromyographic (EMG) signals and the input device 1004 may include an array of EMG electrodes that measure muscle activity indicative of the intended control 1008 of the prosthesis. The at least one ML model 1014 may include a convolutional neural network that computes latent features and a linear discriminant analysis (LDA) classifier that classifies the latent features as one or more gestures.

The predetermined PR control protocol 1010 may be trained with the synthetic noise 1018 in any number or combination of non-limiting examples. For instance, the training data set 1016 may be preconstructed by systematically corrupting a predetermined number of a plurality of channels of raw training signals from the input device 1004. Systematically corrupting the predetermined number of the plurality of channels of the raw training signals may include flatlining, applying Gaussian noise, or a randomized mixture thereof. Augmenting the training data set 1016 can include any variations or features of the system 100 previously described.

II(B). General Non-Limiting Example Method (1100) Associated with the System (1000):

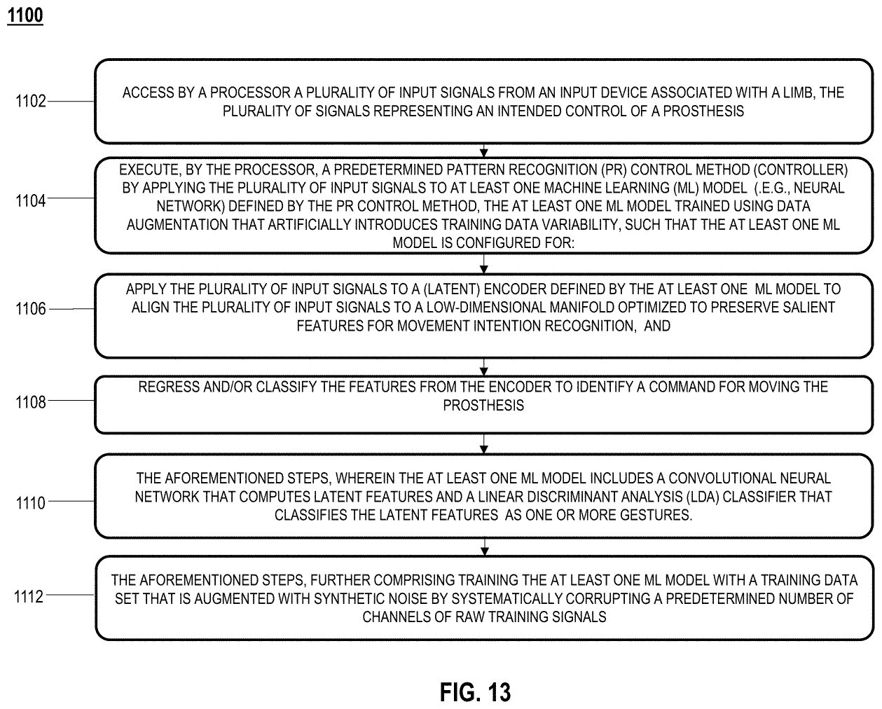

Referring to FIG. 13, to illustrate further aspects of the system 1000, an exemplary method 1100 associated with the system 1000 is shown. Referring to block 1102, a processor (e.g., processor 1002) access a plurality of input signals from an input device associated with a limb. The plurality of input signals represents an intended control of a prosthesis. In some examples, the input device is an EMG array and the plurality of input signals include EMG signals representing muscle activity.

Referring to block 1104, the processor executes a predetermined pattern recognition (PR) control method, controller, or control strategy by applying the plurality of input signals to at least one machine learning model such as a neural network defined by the PR control method. The at least one ML model is trained using data augmentation that artificially introduces training data variability. For example, the at least one ML model is trained using a training data set constructed with synthetic noise, as described herein.

Referring to the blocks 1106 and 1108, executing the at least one ML model as trained, the processor applies the plurality of input signals to a (latent) encoder defined by the at least one ML model to align the plurality of input signals to a low-dimensional manifold optimized to preserve salient features for movement intention recognition. Next, further executing the at least one ML model, classification and/or regression is applied to such salient features from the encoder to identify a comment for moving the prosthesis.

Referring to blocks 1110 and 1112, the at least one ML model can include a convolutional neural network that computes latent (salient) features and a linear discriminant analysis (LDA) classifier that classifies the latent features as one or more gestures. In addition, training the at least one ML model can include utilizing a preconstructed training data set that is augmented with synthetic noise in any of the methods described herein. For example, the training data set can be constructed by systematically corrupting a predetermined number of channels of raw training signals in any manner and combinations thereof described herein.

### III. Example Study & Methods

The following experiment was conducted to evaluate aspects of the system 1000 and was approved by the Northwestern Institutional Review Board. Fourteen individuals with intact limbs (ITL) and seven individuals with below-elbow amputations (AMP, Table 1) participated in this study after providing written informed consent. Due to partial data loss, results from one ITL participant and one AMP participant were excluded from the final analysis.

TABLE 1
Amputee Subject Demographics

| | | | Time Since | | Level Of | DOFs |
|---|---|---|---|---|---|---|
| Subject | Age | Gender | Amputation | | Amputation | Controlled |
| AMP1 | 73 | M | 32 | years | Transradial | 3DOF |
| AMP2 | 33 | M | 5 | years | Wrist | 3DOF |
| | | | | | disarticulation | |
| AMP3 | 65 | M | 6 | years | Transradial | 2DOF |
| AMP4 | 56 | M | 40 | years | Transradial | 2DOF |
| AMP5 | 48 | M | 11 | months | Transradial | 2DOF |
| AMP6 | 19 | M | 10 | months | Transradial | 2DOF |

### Experimental Setup:

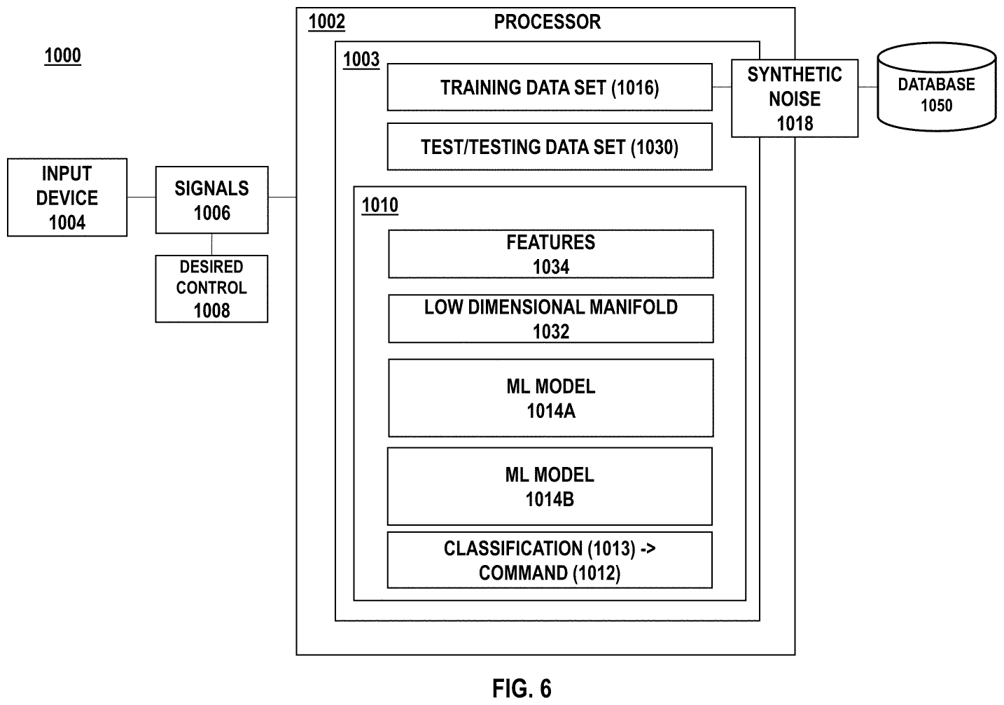

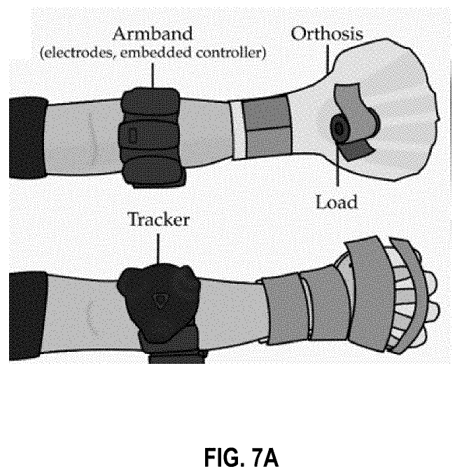

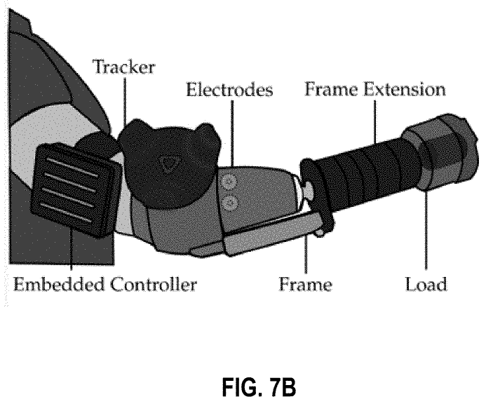

For ITL participants (FIG. 7A), six channels of EMG signals were collected using dry stainless-steel bipolar electrodes (Motion Control Inc.) that were embedded in an adjustable armband. The electrodes were equally spaced around the subject's right arm, with the reference electrode positioned just distal to the olecranon. An HTC Vive tracker was attached to the dorsal side of the armband and used to track the participant's limb position. Participants also wore an orthosis around the wrist and hand to promote isometric contractions that would more closely resemble amputee contractions. Finally, a 400 g weight was attached to the distal end of the orthosis to simulate the weight of a prosthesis.

Due to the unique size and anatomy of each residual limb, dry electrode setups that are not specifically customized for an amputee are prone to electrode liftoff. Hence, wet electrodes for AMP participants to prevent unwanted interface noise (FIG. 7B) were used. Six channels of EMG signals were collected using adhesive Ag/AgCl bipolar electrodes (Bio-Medical Instruments) that were secured under a silicone liner. The electrodes were equally spaced around the subject's residual limb and the reference electrode was placed just distal to the olecranon. An adjustable lightweight frame was fastened around the residual limb and lengthened to match the subject's intact limb length. A 400 g weight was attached to the distal end of the frame to simulate the weight of a prosthesis.

### Data Collection:

All data collection was conducted in an HTC Vive virtual reality environment (FIG. 7C). Each participant collected a training data set and a test data set (1030) during one experimental session. EMG signals were sampled at a rate of 1 kHz, band-pass filtered between 70-300 Hz, and segmented into 200 ms windows in 25 ms increments. In addition to a hardware gain of 2 and a software gain of 1000, there were channel-specific software gains that were customized for each subject. These channel gains were calculated by scaling the signals in each channel to span the output range of −5V to 5V. Although the channel gains were calculated using the training data set alone, they were applied to the training and test data sets.

To collect training data, the subject performed hand and wrist gestures while moving their arm around the workspace. This simple training protocol has been shown to achieve high real-time performance. All ITL subjects and two AMP subjects completed seven gestures (rest, wrist flexion/extension, wrist pronation/supination, hand open/close), corresponding to a 3DOF controller. Based on clinician input and to minimize fatigue, the remaining four AMP participants completed five gestures (rest, wrist pronation/supination, hand open/close), corresponding to a 2DOF controller. Each gesture was held for 2.5 seconds and repeated five times, resulting in 12.5 seconds (500 overlapping windows) of clean training examples per gesture.

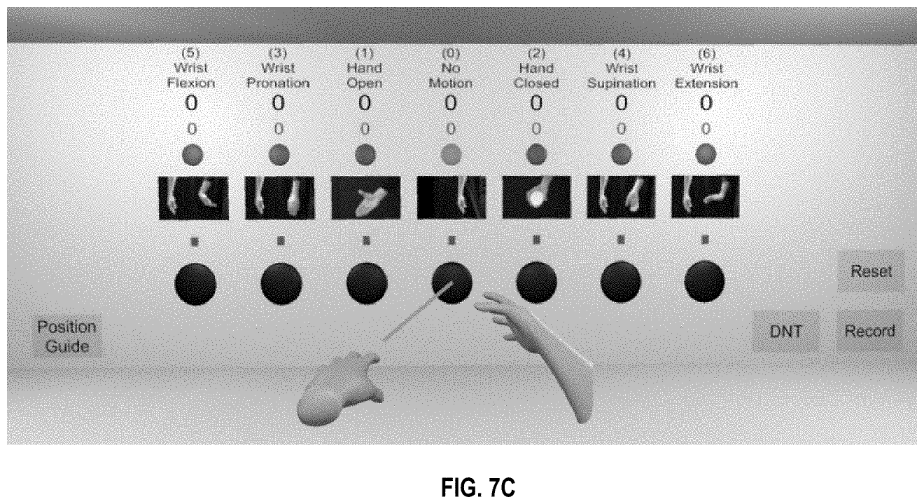

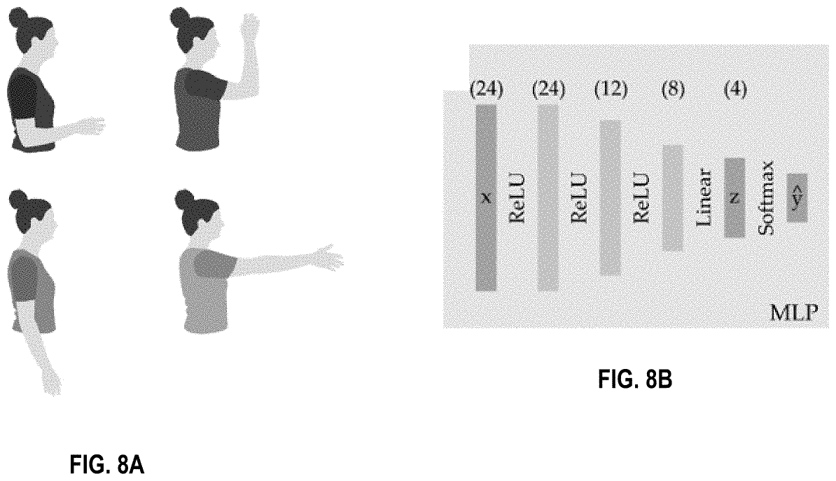

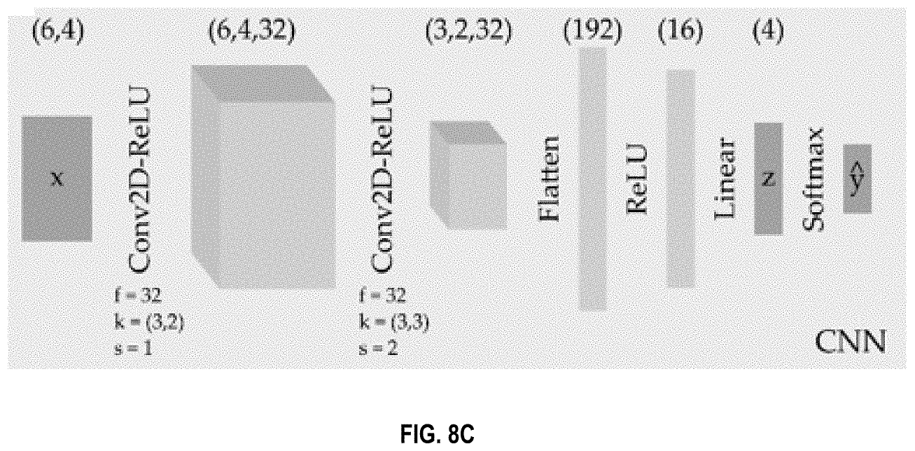

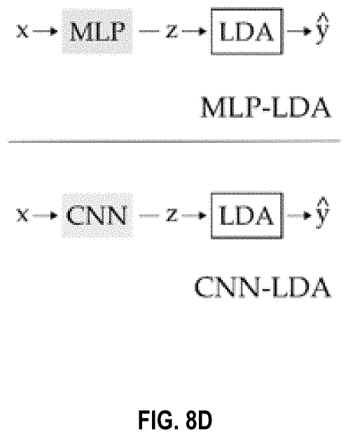

To collect test data, the subject performed the trained hand and wrist gestures in four limb positions (FIG. 8A) Each gesture was held for 2.5 seconds and repeated five times. Therefore, each participant had 50 seconds (2000 overlapping windows) of clean test data for each gesture.

### Offline Analyses:

After EMG data collection, all further analyses were conducted offline on a Windows 10 laptop computer with 16 GB RAM, an Intel Core i7-9850H CPU at 2.60 GHz, and a 4 GB NVIDIA Quadro T1000 GPU. These analyses included training data augmentation, training five control strategies, testing those strategies, and statistical evaluations.

### Training Data Augmentation

An augmented training data set (1016) was constructed set by systematically corrupting up to four channels in copies of the original raw training signals. The augmented data contained one clean copy of the original data and eight noisy copies. Within the eight noisy copies, there were two copies of data that had one noisy EMG channel, two copies that had two noisy channels, two copies that had three noisy channels, and two copies that had four noisy channels. The 12 types of synthetic noise was evenly distributed into all possible channel combinations. These synthetic noise types included flatlining, in which the signal was completely attenuated to 0V, five levels of Gaussian noise centered at 0V (σ=1,2,3,4,5V), five levels of 60 Hz noise (amplitude=1,2,3,4,5V), and a randomized mixture of all noise types.

### Control Strategies/Methods

Five control strategies were trained and evaluated in this study. Before we trained the controllers, four time-domain features were extracted from the training data sets: mean absolute value, waveform length, zero crossings, and slope sign changes.

### Traditional LDA Classifiers

Three control methods were based on the traditional LDA classifier algorithm:
1. Baseline LDA (LDA)—To act as the baseline model, an LDA classifier was trained with the original training data set. This algorithm is used in most clinically available PR systems and therefore demonstrates what current prosthesis users experience.
2. Augmented LDA (LDA+)—To investigate how data augmentation affects the reliability of a standard LDA algorithm, an LDA classifier was trained with the augmented training data set.
3. Adaptive LDA (LDA−)—A fast-retraining LDA classifier was implemented that circumvented signal disturbances by adjusting its LDA weights after removing noisy EMG channels. First, an LDA classifier was trained with the original training data set and stored the class mean and covariance matrices. During classification, elements corresponding to noisy EMG channels were omitted from the mean and covariance matrices and recalculated the LDA weights. Then, noisy EMG channels were removed from the classifier inputs and used the new LDA weights to classify the remaining signals. In practice, this control strategy requires a fault detector to detect noisy signals. However, this step was excluded and instead it was assumed that a perfect fault detector was used. Thus, the LDA− classifier shows the best-case scenario for an adaptive LDA control system. When there were no noisy channels, the LDA− classifier was identical to the baseline LDA classifier.

### Neural Network-Aligned Classifiers

The remaining two control strategies comprised two stages: a latent encoder network that aligned the EMG inputs to a low-dimensional manifold (1032 in FIG. 6) and an LDA classifier that classified these latent variables (FIG. 8D). One control strategy (4) used a multilayer perceptron network (MLP-LDA), while the other control strategy (5) used a convolutional neural network (CNN-LDA). Both models were implemented using Keras 2.3.1 with the Tensorflow backend.

Five-fold cross-validation was used with the augmented training data set to tune each model's hyperparameters. To avoid overlapping training and validation data, each fold corresponded to one gesture repetition. After the hyperparameters were determined, the final models were trained with the entire augmented training data set.

Both networks were trained using the Adam optimization algorithm with a learning rate of 0.001 and mini-batch gradient descent with 30 training epochs and a batch size of 128. To accelerate training time, a minmax scaler was used to standardize the input features between [0,1] and applied batch normalization after each hidden layer. Further details regarding the fourth and fifth control strategies are as follows:
4. Multilayer perceptron-aligned LDA (MLP-LDA)—A fully connected five-layer neural network (FIG. 8B) was trained to take in a 24 by 1 EMG feature vector and output a 4 by 1 latent feature vector z and a predicted gesture label y. The first four hidden layers aligned the EMG inputs to the latent space. ReLU activation functions were applied after the first three layers and a linear activation function after the fourth layer. Based on cross-validation results, it was found that classification accuracy improved as the dimensionality of the latent space increased but began to plateau after a dimensionality of 4. Thus, the latent dimension was set to 4. The weights of the fourth layer were regularized with L1 regularization (λ=10e-5) to encourage sparsity and improve generalization. The last hidden layer in the MLP was a linear classifier that used a softmax activation function to classify the latent features. The network was trained to minimize the categorical cross entropy loss between the predicted class and the ground truth, thus optimizing linear separability between movement classes in the latent space. Since neural network classifiers are prone to overfitting, an LDA classifier was trained with the latent features of the augmented data set and used in tandem with the MLP network to form the MLP-LDA control strategy (FIG. 8D). During classification, the EMG input vectors were passed through the MLP encoder to compute the latent features z, which were then fed to the LDA classifier to obtain gesture predictions. In total, the MLP had 1267 trainable parameters.
5. Convolutional neural network-aligned LDA (CNN-LDA)-A CNN (FIG. 8C) was trained with the same objectives as the MLP: to output a 4 by 1 EMG latent feature vector z and a predicted gesture label y. While the inputs for the previous control strategies were 24 by 1 feature vectors, the CNN input was a 6 by 4 feature matrix, corresponding to the 6 EMG channels and 4 time-domain features. This enabled the 2-dimensional convolutional layers to exploit the spatial relationships between EMG channels and learn more robust latent representations. The first five hidden layers served as the encoder, starting with two convolutional layers with ReLU activation functions. Then, the output of the convolutional layers was flattened before passing it through two sequential layers with a ReLU and a linear activation function, respectively. Thus, the latent encoder modules of the CNN and MLP each had three ReLU functions and one linear function. Like the MLP, the layer preceding the bottleneck was regularized with L1 regularization (λ=10e-5) and the latent space had a dimensionality of 4. The last layer of the CNN classified the latent feature vector z using a softmax activation function. The CNN was trained to minimize the categorical cross entropy loss between the predicted class and the ground truth, once again to encourage linear separability between the class latent representations. Finally, an LDA classifier was trained with the augmented training data set after it was aligned by the CNN. The CNN-LDA control strategy (FIG. 8D) used the CNN encoder to compute latent variables z which were then classified with the LDA classifier. In total, the CNN had 12999 trainable parameters.

### Evaluation

To evaluate control performance and robustness, we calculated the offline classification accuracies of each control strategy on clean and noisy test data. Since it was impractical and challenging to introduce interface noise in a controlled manner during data collection, we constructed noisy test data offline by fusing the original test raw signals with examples from a real noise database.

### Real Noise Database

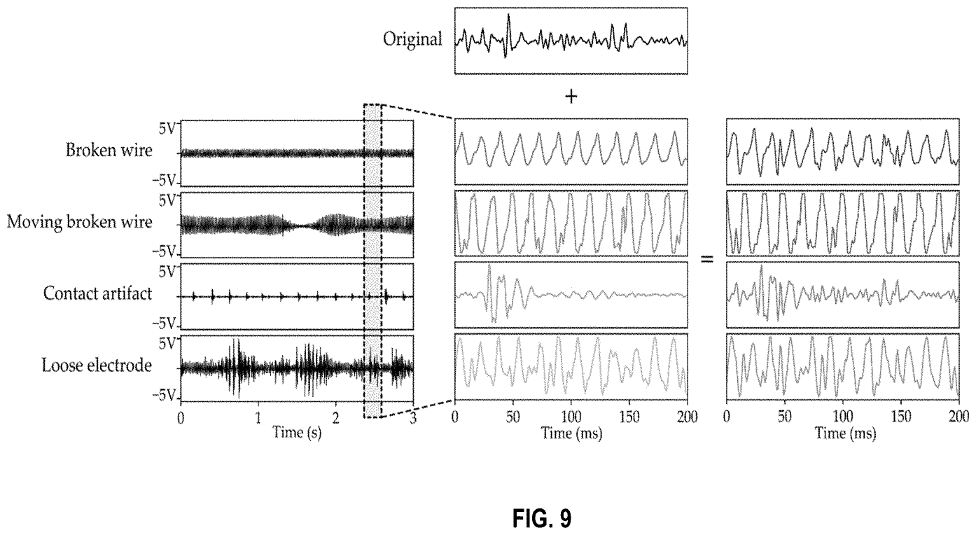

The effects of four noise types were investigated in this study: broken wires, moving broken wires, contact artifacts, and loose electrodes. A database (e.g., database 1050 of FIG. 6) containing 25 seconds (1000 overlapping windows) of each type was collected from one ITL subject (FIG. 9). Since the housing of the armband prevented access to individual electrodes, this database was recorded using the wet electrode setup. Although all six channels were recorded, only the affected noisy channel was stored in the database.

To simulate the broken wire and moving broken wire conditions, one wire was cut at the connection point between the wire and the electrode. For the broken wire condition, the subject maintained a 90-degree angle at the elbow throughout data collection. For the moving broken wire condition, the subject moved their arm around freely in a workspace that contained sources of electrical noise, such as monitors and laptops. For the contact artifact condition, the electrode was tapped approximately every 200 ms. Finally, for the loose electrode condition, the electrode was peeled off and gently shifted around the surface of the subject's skin throughout signal recording.

### Fusion of Test Signals and Real Noise

Four noisy test sets were constructed, each containing a distinct number of noisy EMG channels (1 to 4 channels). Each noisy set started as a copy of the clean test raw signals. Pseudorandomized samples were then systematically superimposed from the real noise database onto the copy, ensuring that all combinations of affected channels and noise types were equally represented. To maintain signal amplification consistency, the subject-specific channel gains were applied to the noise windows according to the channels with which they were being fused. Signals were then truncated to stay within the output range of [−5V, 5V]. Finally, the four time-domain features were extracted from the noisy test signals.

### Statistical Analyses

The statistical analyses were conducted separately for ITL and AMP populations. Linear mixed effects models were used to evaluate the statistical effects of each control algorithm with respect to the baseline LDA method. Initially, a model was fit with classification accuracy as the response variable, the control strategy (LDA, LDA+, LDA−, MLP-LDA, and CNN-LDA), number of noisy electrodes (0-4), and their interactions as fixed factors, and the subject identifier as a random factor. Statistical significance was judged based on a significance level of α=0.05. After observing that all interaction factors were statistically significant (p<0.001), the data were separated by the number of noisy electrodes. These data sets were used to fit five new models that each had the control strategy as a fixed factor and subject identifier as a random factor. The Bonferroni method was used to correct for multiple comparisons.

### Results

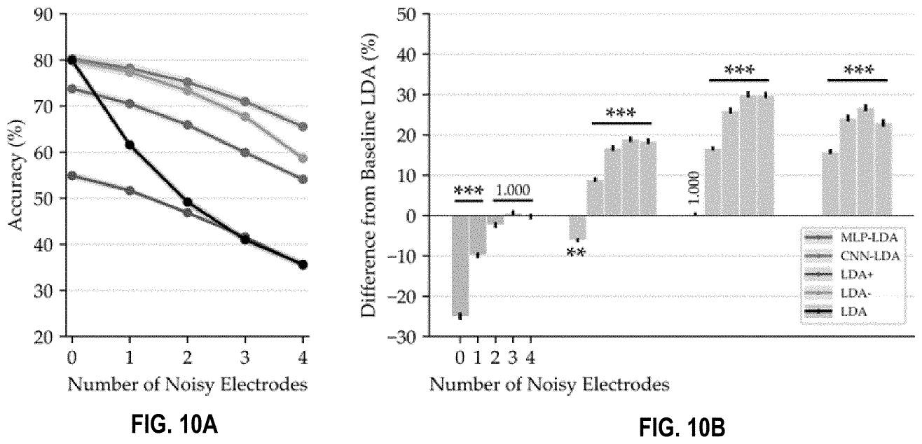

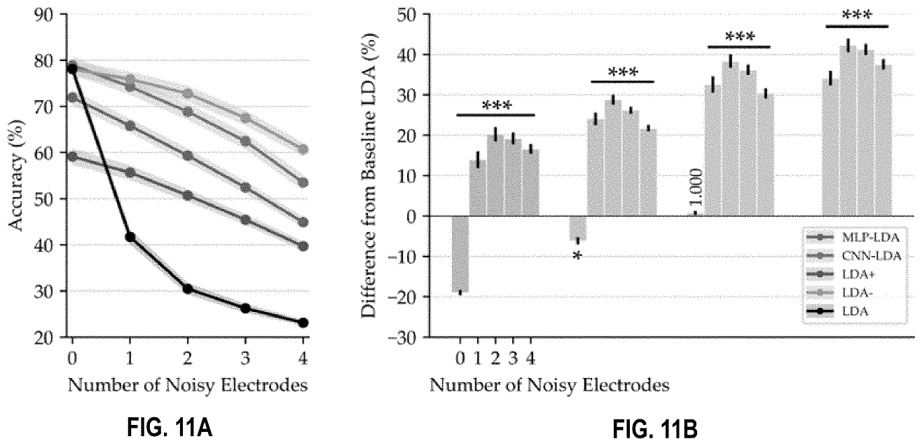

The classification accuracies of the five control strategies and summary of the statistical models are shown in FIGS. 10A-10B, and FIGS. 11A-11B.

### Interface Noise Degrades LDA Accuracy

Under standard conditions, the baseline LDA classifier decoded gestures with average accuracies of 79.92±1.14% and 78.10±1.66% for ITL and AMP participants respectively. When noise was present in just one channel, the accuracies dropped to 61.55±0.98% and 41.74±1.62%, demonstrating that a minor change in input signals can have a large impact on control performance, particularly for AMP subjects. As the number of corrupted channels increased, the accuracy continued to decrease.

### Data Augmentation May Increase Noise Tolerance, but at a Cost

For the AMP population, augmenting the training data set with synthetic noise increased the robustness of an LDA classifier. Compared to the 36.36% drop in baseline LDA accuracy between the noiseless and single channel conditions, the LDA+ accuracy decreased by only 4.19%. Consequently, the LDA+ control strategy significantly outperformed the baseline LDA control strategy for all noisy conditions (p<0.001). However, with an accuracy of 59.12±1.79%, the LDA+ algorithm was also significantly worse at classifying clean signals compared to the baseline LDA algorithm (p<0.001).

The trends for the LDA+ classifier were different in ITL subjects. Its outcomes were significantly worse compared to those of the baseline LDA classifier for the noiseless and single channel noise conditions (p<0.001), but not significantly different for the remaining noisy conditions.

### Neural Network Models Outperform Non-Adaptive LDA Algorithms

Generally, the neural network-aligned methods improved overall outcomes compared to all non-adaptive LDA methods. Across all test sets, MLP-LDA had average accuracies of 64.84% (ITL) and 58.88% (AMP), which were 18.72% and 7.73% higher than LDA+ accuracies. MLP-LDA also improved classification of noisy signals by 15.77% (ITL) and 25.22% (AMP) compared to the baseline LDA classifier. However, there were statistically significant drops in accuracy on clean signals (ITL: 6.13%, AMP: 6.18%). Thus, at best, MLP-LDA had a 73.78±0.99% accuracy for ITL subjects and a 71.92±1.72% accuracy for AMP subjects.

In contrast, the CNN-LDA control strategy significantly improved classification of noisy EMG signals (p<0.001) without decreasing accuracy on clean EMG signals (p=1.000). CNN-LDA classified normal signals with an accuracy of 80.25±1.21% (ITL) and 78.91±1.89% (AMP), exhibiting the best performances across all five control strategies. Unsurprisingly, these accuracies decreased as noise was introduced into the system. However, CNN-LDA scored 65.52±0.90% (ITL) and 53.49±1.71% (AMP) with four noisy channels, meaning that at its worst, it still performed better than baseline LDA did with only one noisy channel. Therefore, CNN-LDA was the most accurate and robust non-adaptive control strategy.

### CNN-LDA Eliminates Need for Controller Adaptation

Overall, CNN-LDA and LDA− achieved similar performances. For AMP participants, the decrease in accuracy from LDA- to CNN-LDA for noisy data ranged from 1.61% for single-channel noise to 7.20% for four-channel noise. For ITL subjects, CNN-LDA accuracies surpassed LDA−, with improvements ranging from 0.79% for single-channel noise and 6.87% for four channel noise. Although the study did not statistically compare these differences, it is unlikely that they would cause significant clinical impact. Thus, CNN-LDA was functionally equivalent to an adaptive LDA control system with a perfect fault detector.

### LDA Components Illustrate Performance Differences

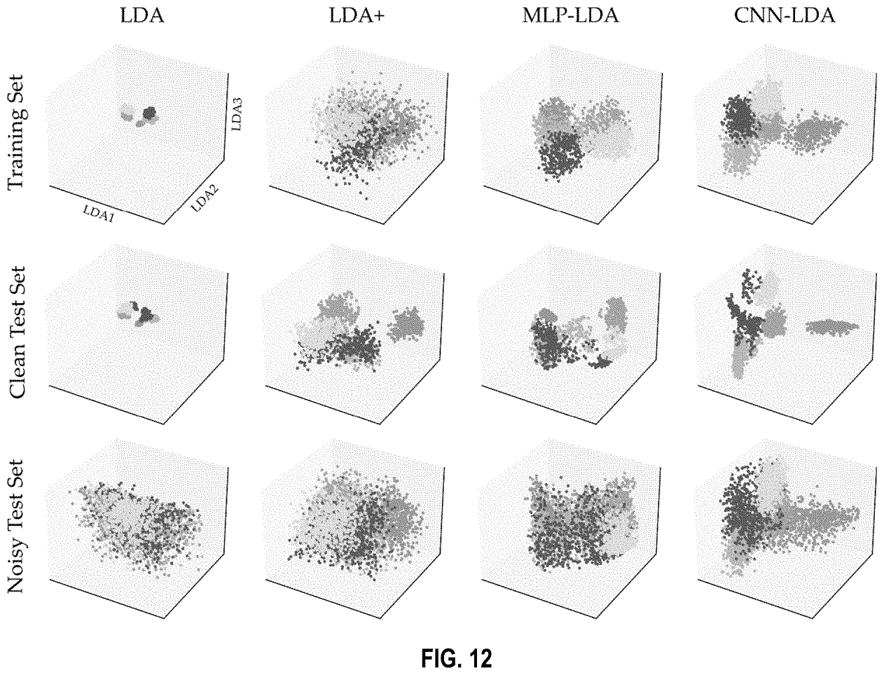

To visualize the underlying mechanisms of the non-adaptive classifiers, we plotted the first three LDA components of the clean training data, clean test data, and noisy test data for subject AMP5 (FIG. 12). For baseline LDA and LDA+, the study employed the projection matrices from the trained LDA classifiers to reduce the high-dimensional input features. For MLP-LDA and CNN-LDA, the input feature data were aligned to their latent manifolds through the MLP and CNN encoders before applying the projection matrices from their corresponding LDA classifiers.

CNN-LDA was the only control strategy that maintained separable clusters across all three data sets, explaining why it was able to effectively classify both clean and noisy signals. In contrast, LDA and MLP-LDA clusters lost their separability when noise was introduced, thus depicting their lower noise tolerance. The LDA+ clean test set clusters did not match the clean training set clusters; therefore, the decision boundaries computed from the training set were not able to delineate the gestures in clean test data set.

### Neural Network Methods Require Longer Processing Times

Table 2 below shows the processing times of each control method to assess their practicality in clinical settings. Notably, the initial training processing times for the neural network methods, which included data augmentation time, were substantially longer than those for the traditional LDA methods. Likewise, the classification processing times were longer for the neural network methods. However, at ˜5 ms, this was still shorter than the EMG window sampling increments (25 ms), indicating that a real-time implementation is plausible.

TABLE 2
Controller Processing Times

| | TRAINING PROCESSING | CLASSIFICATION |
|---|---|---|
| | TIME | PROCESSING TIME |
| LDA | 0.0259 s ± 1.37 ms | 0.00659 ms ± 64.4 ns |
| LDA+ | 1.22 s ± 48.6 ms | 0.00665 ms ± 68.8 ns |
| LDA− | 0.0259 s ± 1.37 ms | 0.328 ms ± 4.02 us |
| MLP-LDA | 51.3 s ± 2.82 s | 5.54 ms ± 38.1 us |
| CNN-LDA | 47.8 s ± 3.12 s | 5.75 ms ± 38.7 us |

### Discussion

Clinical PR-based myoelectric control systems often encounter error-producing signal noise stemming from EMG interface instabilities. Previous works have reduced these errors with additional processing steps such as signal filtering, noise detection, and controller recalibration or adaptation. It is believed that this is the first study presenting a PR control strategy that is inherently resistant to various types of multichannel interface noise. Using examples of real interface noise, the reliability of LDA-based classifiers that employed data augmentation and deep learning techniques were evaluated.

In general, it was found that the adverse effects of interface noise were more severe in the amputee population than the intact limb population. One potential reason is that intact limb muscle contractions often have larger amplitudes compared to residual limb contractions. Therefore, noisy intact limb EMG signals have higher signal-to-noise ratios (SNR), leading to better classification rates.

The high SNR in intact limb participants may also explain why augmenting the training data set with artificial noise did not improve overall outcomes for the augmented LDA classifier. The augmented data set contained various levels of signal corruption that spanned the output range of the EMG channels. Consequently, it had a wide range of SNRs as opposed to the relatively high SNRs of the noisy test set. Thus, the augmented LDA classifier was trained on distributions that were not representative of the testing data, causing it to perform poorly.

The results also showed that manifold alignment facilitated by neural networks produced impactful performance gains. This suggests that nonlinear transformations are crucial to extracting useful discriminative structures within EMG signals. Furthermore, the CNN outperformed the multilayer perceptron, emphasizing that the spatial relationships between channels are valuable and should be preserved and leveraged for better control. For the intact limb population, the CNN-LDA method was also more accurate than the adaptive LDA method. This exemplified an important advantage of the CNN-LDA method: it retains discriminative features in the noisy channels that would normally be discarded by recalibration algorithms. Notably, CNN-LDA was able to classify signals with real interface noise even though the augmented training data set only contained synthetic noise, highlighting the generalizability of the deep learning model.

Ultimately, the CNN-LDA control strategy is an attractive solution to the interface noise problem, as it was shown to be an non-adaptive method that improved noise tolerance without reducing accuracy on normal signals. In reality, disturbances would most likely occur in one or two EMG channels. For these cases, CNN-LDA obtained classification accuracies (ITL: >75.19%, AMP: >68.83%) that would suggest effective real-time control.

The clinical implications of the CNN-LDA controller are that patients would be able to maintain good control of their prosthesis across common scenarios that produce intermittent noise (e.g., electrode liftoff, contact artifacts) or continuous noise (e.g., broken wires). Moreover, it does not require a noise detector, which can be inaccurate, or recalibration, which adds a processing step. This method is highly beneficial for amputee users, as their low EMG SNR renders their clinical LDA systems unusable in the presence of noise.

However, there are some practical limitations to the CNN-LDA controller. For example, its training and execution processing time is slower than traditional LDA methods. Also, the memory requirements of the model may hinder its implementation on a prosthesis microcontroller. Lastly, it is difficult to quickly retrain a black box model such as the CNN-LDA. If the user wanted to recalibrate a single gesture, the entire backpropagation process would have to be repeated.

### Considerations & Additional Contemplated Features

The main limitation of the foregoing study is that the controllers were evaluated offline. Real-time experiments should be conducted to investigate the effects of user adaptation and provide a more realistic evaluation of prosthesis controllability. Additionally, while commercial systems typically include eight EMG channels, the experimental setup only used six EMG channels. The implication is that each noisy signal has a greater influence on classification accuracy. Thus, we expect the performance to be better with clinical setups that have eight EMG channels.

Four amputee subjects only completed enough gestures for a 2DOF controller instead of 3DOF controller. These algorithms were also limited to sequential control but would be more impactful if extended to simultaneous control. To facilitate practical implementation of the CNN-LDA, network architecture and adjust hyperparameters may be further minimized to balance controller performance and processing time. In addition to conducting real-time experiments with a physical prosthesis, it would be useful to investigate the controller's robustness to donning/doffing and its long-term stability.

### Exemplary Computing Device:

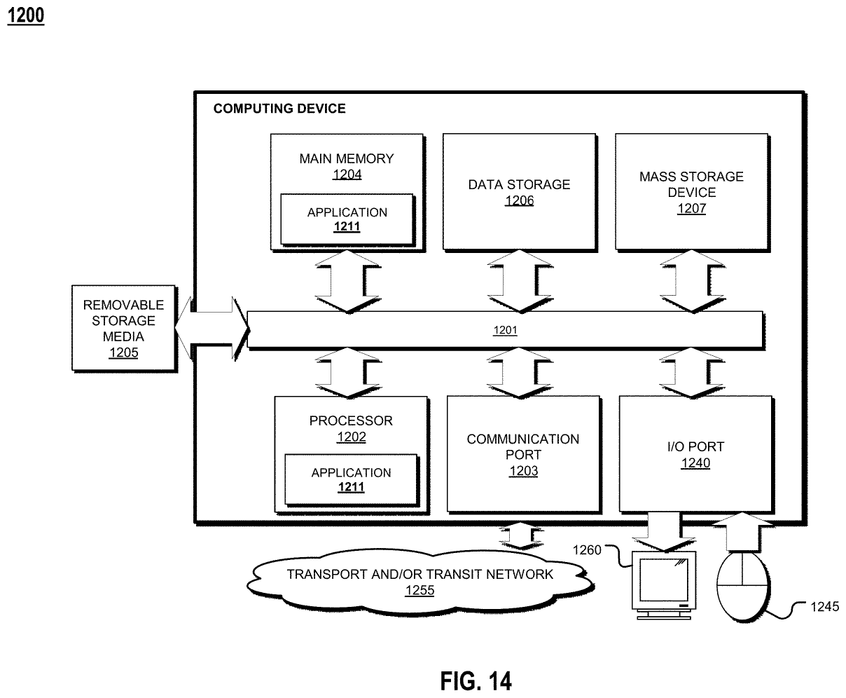

Referring to FIG. 14, a computing device 1200 is illustrated which may be configured, via one or more of an application 1211 or computer-executable instructions, to execute functionality described herein. More particularly, in some embodiments, aspects of the deep learning techniques using augmented training data for PR myoelectric control described herein may be translated to software or machine-level code, which may be installed to and/or executed by the computing device 1200 such that the computing device 1200 is configured to execute functionality described herein. It is contemplated that the computing device 1200 may include any number of devices, such as personal computers, server computers, hand-held or laptop devices, tablet devices, multiprocessor systems, microprocessor-based systems, set top boxes, programmable consumer electronic devices, network PCs, minicomputers, mainframe computers, digital signal processors, state machines, logic circuitries, distributed computing environments, and the like.

The computing device 1200 may include various hardware components, such as a processor 1202, a main memory 1204 (e.g., a system memory), and a system bus 1201 that couples various components of the computing device 1200 to the processor 1202. The system bus 1201 may be any of several types of bus structures including a memory bus or memory controller, a peripheral bus, and a local bus using any of a variety of bus architectures. For example, such architectures may include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

The computing device 1200 may further include a variety of memory devices and computer-readable media such as a storage device 1207 that includes removable/non-removable media (1205) and volatile/nonvolatile media and/or tangible media, but excludes transitory propagated signals. Computer-readable media may also include computer storage media and communication media. Computer storage media includes removable/non-removable media and volatile/nonvolatile media implemented in any method or technology for storage of information, such as computer-readable instructions, data structures, program modules or other data, such as RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information/data and which may be accessed by the computing device 1200. Communication media includes computer-readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. For example, communication media may include wired media such as a wired network or direct-wired connection and wireless media such as acoustic, RF, infrared, and/or other wireless media, or some combination thereof. Computer-readable media may be embodied as a computer program product, such as software stored on computer storage media.

The data storage 1206 may also include other removable/non-removable, volatile/nonvolatile computer storage media. For example, the data storage 1206 may be: a hard disk drive that reads from or writes to non-removable, nonvolatile magnetic media; a magnetic disk drive that reads from or writes to a removable, nonvolatile magnetic disk; a solid state drive; and/or an optical disk drive that reads from or writes to a removable, nonvolatile optical disk such as a CD-ROM or other optical media. Other removable/non-removable, volatile/nonvolatile computer storage media may include magnetic tape cassettes, flash memory cards, digital versatile disks, digital video tape, solid state RAM, solid state ROM, and the like. The drives and their associated computer storage media provide storage of computer-readable instructions, data structures, program modules, and other data for the computing device 1200.

A user may enter commands and information through a user interface via an I/O port 1240 (displayed via a monitor 1260) by engaging input devices 1245 such as a tablet, electronic digitizer, a microphone, keyboard, and/or pointing device, commonly referred to as mouse, trackball or touch pad. Other input devices 1245 may include a joystick, game pad, satellite dish, scanner, or the like. Additionally, voice inputs, gesture inputs (e.g., via hands or fingers), or other natural user input methods may also be used with the appropriate input devices, such as a microphone, camera, tablet, touch pad, glove, or other sensor. These and other input devices 1245 are in operative connection to the processor 1202 and may be coupled to the system bus 1201 but may be connected by other interface and bus structures, such as a parallel port, game port or a universal serial bus (USB). The monitor 1260 or other type of display device may also be connected to the system bus 1201. The monitor 1260 may also be integrated with a touch-screen panel or the like.

The computing device 1200 may be in operable communication with one or more networks 1255, implemented in a networked or cloud-computing environment using logical connections of a network interface (communication port 1203) to one or more remote devices, such as a remote computer. The remote computer may be a personal computer, a server, a router, a network PC, a peer device or other common network node, and typically includes many or all of the elements described above relative to the computing device 1200. The logical connection may include one or more local area networks (LAN) and one or more wide area networks (WAN) but may also include other networks. Such networking environments are commonplace in offices, enterprise-wide computer networks, intranets and the Internet.

When used in a networked or cloud-computing environment, the computing device 1200 may be connected to a public and/or private network through the network interface. In such embodiments, a modem or other means for establishing communications over the network is connected to the system bus 1201 via the network interface or other appropriate mechanism. A wireless networking component including an interface and antenna may be coupled through a suitable device such as an access point or peer computer to a network. In a networked environment, program modules depicted relative to the computing device 1200, or portions thereof, may be stored in the remote memory storage device.

Certain embodiments are described herein as including one or more modules. Such modules are hardware-implemented, and thus include at least one tangible unit capable of performing certain operations and may be configured or arranged in a certain manner. For example, a hardware-implemented module may comprise dedicated circuitry that is permanently configured (e.g., as a special-purpose processor, such as a field-programmable gate array (FPGA) or an application-specific integrated circuit (ASIC)) to perform certain operations. A hardware-implemented module may also comprise programmable circuitry (e.g., as encompassed within a general-purpose processor or other programmable processor) that is temporarily configured by software or firmware to perform certain operations. In some example embodiments, one or more computer systems (e.g., a standalone system, a client and/or server computer system, or a peer-to-peer computer system) or one or more processors may be configured by software (e.g., an application or application portion) as a hardware-implemented module that operates to perform certain operations as described herein.

Accordingly, the term “hardware-implemented module” encompasses a tangible entity, be that an entity that is physically constructed, permanently configured (e.g., hardwired), or temporarily configured (e.g., programmed) to operate in a certain manner and/or to perform certain operations described herein. Considering embodiments in which hardware-implemented modules are temporarily configured (e.g., programmed), each of the hardware-implemented modules need not be configured or instantiated at any one instance in time. For example, where the hardware-implemented modules comprise a general-purpose processor configured using software, the general-purpose processor may be configured as respective different hardware-implemented modules at different times. Software may accordingly configure the processor 1202, for example, to constitute a particular hardware-implemented module at one instance of time and to constitute a different hardware-implemented module at a different instance of time.

Hardware-implemented modules may provide information to, and/or receive information from, other hardware-implemented modules. Accordingly, the described hardware-implemented modules may be regarded as being communicatively coupled. Where multiple of such hardware-implemented modules exist contemporaneously, communications may be achieved through signal transmission (e.g., over appropriate circuits and buses) that connect the hardware-implemented modules. In embodiments in which multiple hardware-implemented modules are configured or instantiated at different times, communications between such hardware-implemented modules may be achieved, for example, through the storage and retrieval of information in memory structures to which the multiple hardware-implemented modules have access. For example, one hardware-implemented module may perform an operation, and may store the output of that operation in a memory device to which it is communicatively coupled. A further hardware-implemented module may then, at a later time, access the memory device to retrieve and process the stored output. Hardware-implemented modules may also initiate communications with input or output devices.

Computing systems or devices referenced herein may include desktop computers, laptops, tablets e-readers, personal digital assistants, smartphones, gaming devices, servers, and the like. The computing devices may access computer-readable media that include computer-readable storage media and data transmission media. In some embodiments, the computer-readable storage media are tangible storage devices that do not include a transitory propagating signal. Examples include memory such as primary memory, cache memory, and secondary memory (e.g., DVD) and other storage devices. The computer-readable storage media may have instructions recorded on them or may be encoded with computer-executable instructions or logic that implements aspects of the functionality described herein. The data transmission media may be used for transmitting data via transitory, propagating signals or carrier waves (e.g., electromagnetism) via a wired or wireless connection.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A system that leverages data augmentation and deep learning to improve noise tolerance of myoelectric pattern recognition, comprising:
an electromyography (EMG) array operable for measuring EMG data; and
a processor in operative communication with the EMG array, the processor executing a controller defining:
a feature extraction module configured to extract a plurality of features from an original set and a corrupted set of EMG data and produce a plurality of feature windows; and
a neural network configured to reconstruct and classify each feature window of the plurality of feature windows produced by the feature extraction module, the neural network comprising:
an encoder module configured to project an input feature window of the plurality of feature windows to a latent distribution, wherein a latent variable is sampled for the input feature window from the latent distribution; and
a classifier module configured to predict movement classes associated with the input feature window of the plurality of feature windows from the latent variable, the classifier module trained using training data augmentation.

2. The system of claim 1, further comprising:
a signal corruptor module configured to corrupt EMG data measured by the EMG array, wherein each channel of the EMG data is corrupted to produce the original set of EMG data and the corrupted set of EMG data.

3. The system of claim 1, further comprising:
a decoder module configured to generate a reconstructed set of EMG data associated with the input feature window of the plurality of feature windows from the associated latent variable.

4. The system of claim 1, further comprising:
a data separation module operable for separating the EMG data into a training subset and a testing subset.

5. The system of claim 2, wherein the original set includes a plurality of EMG channels and wherein the corrupted set includes a plurality of corrupted copies of the original set, wherein each copy of the plurality of corrupted copies includes at least one corrupted segment.

6. The system of claim 5, wherein at least one corrupted segment defines powerline interference superimposed onto the original signal on one or more channels

7. The system of claim 1, wherein the powerline interference superimposed is 50 hz to 60 hz.

8. The system of claim 1, wherein the neural network is a supervised denoising variational autoencoder, and the neural network minimizes a mean squared error between an original set of EMG data and a reconstructed set of EMG data.

9. The system of claim 1, wherein the neural network minimizes a Kullback-Leibler divergence between the latent distribution and a standard normal distribution.

10. The system of claim 1, wherein the neural network minimizes a cross-entropy loss between a set of ground truth class labels and a set of predicted movement class labels associated with the movement classes predicted by the classifier module.

11. A method for improved interface noise tolerance with pattern recognition controllers, comprising:
accessing, by a processor, a plurality of input signals from an input device associated with a limb, the plurality of signals representing an intended control of a prosthesis; and
executing, by the processor, a predetermined pattern recognition controller by applying the plurality of input signals to at least one ML model, the at least one ML model trained using data augmentation that artificially introduces training data variability, the at least one ML model configured for:
applying the plurality of input signals to a latent encoder defined by the at least one ML model to align the plurality of input signals to a low-dimensional manifold optimized to preserve salient features for movement intention recognition, and
identifying a command for moving the prosthesis from the salient features.

12. The method of claim 11, wherein the at least one ML model includes a convolutional neural network that computes latent features and a linear discriminant analysis (LDA) classifier that classifies the latent features as one or more gestures.

13. The method of claim 11, further comprising training the at least one ML model using a training data set that is augmented with synthetic noise.

14. The method of claim 13, wherein the at least one ML model includes an LDA classifier that is trained with latent features of the training data set, the training data set being augmented with the synthetic noise.

15. The method of claim 13, further comprising constructing the training data set by systematically corrupting a predetermined number of a plurality of channels of raw training signals from the input device.

16. The method of claim 15, wherein systematically corrupting the predetermined number of the plurality of channels of the raw training signals includes flatlining, applying Gaussian noise, or a randomized mixture thereof.

17. The method of claim 11, wherein the plurality of signals includes electromyographic (EMG) signals and the input device includes an array of EMG electrodes that measure muscle activity indicative of the intended control of the prosthesis.

18. A device for implementing pattern recognition using data augmentation for improved noise tolerance, comprising:
an input device that generates a plurality of signals, the plurality of signals representing an intended control of a prosthesis; and
a processor in operable communication with the input device, the processor executing a predetermined pattern recognition controller that applies the plurality of input signals to at least one ML model, the at least one ML model trained using a training data set augmented with synthetic noise, the at least one ML model configured to:
align the plurality of input signals to a low-dimensional manifold defining features, and
identify from the features a command for moving the prosthesis.

19. The device of claim 18, wherein the at least one ML model includes an LDA classifier that is trained with latent features of the training data set, the training data set constructed by systemic corruption of a predetermined number of a plurality of channels of raw training signals from the input device.

20. The device of claim 18, wherein the plurality of signals includes electromyographic (EMG) signals and the input device includes an array of EMG electrodes that measure muscle activity indicative of the intended control of the prosthesis.
